(19) United States
(12) Patent Application Publication
(10) Pub. No.: US 2012/0045528 A1
(43) Pub. Date: Feb. 23, 2012

(54) USE OF N20 GAS FOR TREATING CHRONIC PAIN

(76) Inventors: Baptise Bessiere, Issy Les Moulineaux (FR); Guy Simonnet, Bordeaux (FR)

(21) Appl. No.: 13/318,086

(22) PCT Filed: Apr. 12, 2010
(86) PCT No.: PCT/FR2010/050697

§ 371 Date: Oct. 28, 2011

(30) Foreign Application Priority Data
Apr. 29, 2009 (FR) 0952807

(51) Int. Cl. A61K 33/00; A61P 25/04; A61P 25/00

(52) U.S. Cl. 424/718

(57) ABSTRACT

The invention relates to a gas mixture that contains nitrous oxide (N₂O) for use as a breathable drug for the treatment of chronic pain in a mammal, in particular a pain from neuropathic, iatrogenic, dysfunctional or inflammatory origin, in particular in humans. The proportion of N₂O ranges from 15 to 45 vol. %. The gas mixture is administered for a sufficient duration for obtaining a delayed pain hypersensitivity reduction that can be observed at least 6 hours after the end of the inhalation of the gas mixture by said mammal. The pain hypersensitivity is selected from allodynias and hyperalgesias.

### CROSS REFERENCE TO RELATED APPLICATIONS

This application is a 371 of International PCT Application PCT/FR2010/050697, filed Apr. 12, 2010, which claims priority to French Application 0952807, filed Apr. 29, 2009, the entire contents of which are incorporated herein by reference.

The invention relates to the use of nitrous oxide (N₂O) at low concentrations, typically at less than 50% by volume, in the treatment of chronic pain, mainly of neuropathic origin, i.e. linked to nerve damage, of dysfunctional origin, of inflammatory origin or of iatrogenic origin, i.e. following the taking of medicaments, for example medicaments used in chemotherapy.

In the general population, it is estimated that one individual in six suffers from chronic pain, as described by the document Breivik, H. et al, Survey of chronic pain in Europe: prevalence, impact on daily life, and treatment. Eur. J. Pain 10, 287-333 (2006).

Among chronic pain, neuropathic pain is considered to be the most severe and the most persistent.

Neuropathic pain occurs following damage to or dysfunction of the nervous system, which can be:
either of peripheral origin, for example damage or irritation, of traumatic, toxic, metabolic, ischemic, immunoallergic or infectious origin, to or of the peripheral nerves,
or of central origin, for example damage to or irritation of the spinal cord or of the encephalon.

Neuropathic pain does not respond to the usual analgesics, such as nonsteroidal anti-inflammatories, paracetamol or salicylated compounds, and is often not very sensitive to opioid analgesics.

At the neurobiological level, hypersensitivity to pain, i.e. hyperalgesia or allodynia, following nerve damage (neuropathic pain) is not only due to an excess of nociception, but involves pain sensitization processes, as summarized by the documents T. J. Coderre, et al. Peripheral and central hyperexcitability: differential signs and symptoms in persistent pain, Behav. Brain Sci. 20, 404-419, (1997); and C. J. Woolf et al., Neuropathic pain: aetiology, symptoms, mechanisms, and management, Lancet 353, 1959-1964 (1999).

The key role played by the NMDA (N-methyl-D-aspartate) receptor in the pain sensitization process makes this receptor a therapeutic target of choice in the treatment of chronic pain mainly of neuropathic origin, as described by the document C. G. Parsons, NMDA receptors as targets for drug action in neuropathic pain, Eur. J. Pharmacol. 429, 71-78 (2001). Thus, the clinical efficacy of NMDA receptor antagonists, such as ketamine, amantadine or memantine, has been described, in particular by the document Chizh, B. A. et al, NMDA antagonists and neuropathic pain—multiple drug targets and multiple uses, Curr. Pharm. Des 11, 2977-2994 (2005).

However, the adverse effects of the current NMDA receptor antagonists, such as memory problems, psychodysleptic effects, ataxia and poor motor coordination, largely offset the expected benefits and prohibit wide or sustained use thereof, as explained by the documents J. A. Kemp et al, NMDA receptor pathways as drug targets, Nat Neurosci. 5 Suppl. 1039-1042 (2002) and B. A. Chizh et al., NMDA receptor antagonists as analgesics: focus on the NR2B subtype, Trends Pharma. Sci. 22, 636-642 (2001).

Starting from that point, a problem which arises is that of having a curative treatment or medicament which acts at the level of the NMDA receptor while showing efficacy in the treatment of chronic pain, in particular pain of neuropathic origin, of dysfunctional origin, of inflammatory origin or of iatrogenic origin, but generating, moreover, few or no adverse effects, i.e. adverse effects which are limited compared with those that exist when the current NMDA receptor antagonists are used.

One solution to this problem is a gas mixture containing a proportion of nitrous oxide (N₂O) between 15% and 45% by volume for use as an inhalable medicament for the curative treatment of chronic pain in a mammal, the gas mixture being administered for a period of time sufficient to obtain a delayed pain hypersensitivity reduction, also called delayed anti-sensitization effect, that can be observed at least 6 hours after the end of the inhalation of the gas mixture by said mammal.

As appropriate, the gas mixture of the invention may comprise one or more of the following characteristics:
the pain hypersensitivity is selected from allodynia and hyperalgesia;
the mammal is selected from a human being, namely a man or a woman, including children and newborns;
the proportion of N₂O in the gas mixture is between 15% and 40% by volume;
the proportion of N₂O in the gas mixture is at least 20% by volume, preferably at least 25% by volume;
the chronic pain is of neuropathic origin, of dysfunctional origin, of inflammatory origin or of iatrogenic origin;
the gas mixture is administered for a period of time sufficient to obtain an analgesic effect for at least a part of the period of administration by inhalation of the gas mixture and a delayed anti-sensitization effect after the end of the inhalation of the gas mixture by said mammal;
the gas mixture is administered for a period of time of at least 20 min, preferably at least 30 min, preferably at least 40 min, advantageously at least 1 h;
the gas mixture is administered for a period of time sufficient to obtain a delayed anti-sensitization effect that occurs and that can be observed at least 12 hours after the end of the inhalation of the gas mixture;
the gas mixture is administered for a period of time sufficient to obtain a delayed pain hypersensitivity reduction that occurs and that can be observed at least 24 hours after the end of the inhalation of the gas mixture;
the gas mixture also contains between 20% and 60% by volume of oxygen, preferably between 20% and 50% by volume of oxygen;
the gas mixture is made up of 15% to 45% by volume of N₂O and at least 20% by volume of oxygen;
the gas mixture optionally contains one or more other gases selected from xenon, argon, helium, neon, krypton and nitrogen, preferably in a content by volume less than that of the N₂O present in the mixture;
the gas mixture is made up of 15% to 45% by volume of N₂O and at least 20% by volume of oxygen, and also contains xenon, argon, helium, neon, krypton or nitrogen, in particular nitrogen or xenon;
the gas mixture is made up of 15% to 45% by volume of N₂O and of 21% to 60% by volume of oxygen, with the rest being nitrogen;
the pain of neuropathic origin is selected from post-shingles pain, pain caused by diabetic neuropathies, central pain, post-stroke pain, pain caused by multiple sclerosis, neuropathic pain caused by medullary traumas, neuropathic pain related to AIDS or to the treatment thereof, cancer-related neuropathic pain, chemotherapy-related neuropathic pain, neuropathic pain related to a surgical act which has damaged a nerve pathway and post-operative neuropathic pain;
the pain of dysfunctional origin is related to fibromyalgia, to irritable bowel syndrome or to cephalalgia;
the pain of inflammatory origin is related to arthrosis, arthritis or a herniated disk;
the N₂O can, moreover, be administered in combination with an analgesic included in the steps of the WHO (World Health Organization), namely:
   The step I analgesics, also called “peripheral analgesics” or “non-morphine analgesics”, which have the lowest analgesic potency, for example paracetamol, aspirin, nonsteroidal anti-inflammatories, etc.
   The step II analgesics termed “central” or “weak morphine” analgesics, since they are active on pain perception at the cerebral level. They are used alone, for example tramadol, or in combination with step I analgesics, for example codeine-paracetamol.
   The step III analgesics which group together strong morphine agonists and antagonist agonists.

In other words, the invention also relates to the use of a gas mixture containing nitrous oxide (N₂O) according to the invention, i.e. between 15% and 45% by volume of N₂O as described above, for producing an inhalable medicament for the curative treatment of chronic pain in a mammal, in particular in a human being, the gas mixture being administered for a period of time sufficient to obtain a delayed pain hypersensitivity reduction, i.e. a delayed anti-sensitization effect, that can be observed at least 6 hours after the end of the inhalation of the gas mixture by said mammal.

The present invention will now be understood more clearly by virtue of the following description and the examples which follow, which are given purely by way of illustration.

In the context of the present invention, the nitrous oxide (N₂O) is used at a concentration or concentrations preferentially between 15% and 45% by volume in order to reduce, in a sustained manner, chronic pain in a human being, i.e. a man, a woman, a child or an infant, mainly pain of neuropathic, dysfunctional, inflammatory or iatrogenic origin.

The gas mixture containing nitrous oxide (N₂O) that can be used as an inhalable medicament for the curative treatment of chronic pain in a patient suffering from such chronic pain, in particular of neuropathic origin, can be administered by inhalation in the context of a therapeutic treatment method for a patient suffering from chronic pain.

The gas mixture of the invention can be used in the context of a therapeutic treatment method in which said N₂O-based gas mixture is administered by inhalation, for example by means of a breathing mask which is either directly connected to a source of N₂O at the required concentration, for example a ready-to-use gas cylinder, or else to the outlet of a gas mixer fed with several gas sources (O₂, N₂O, etc.) so as to obtain the desired mixture; or connected to a respiratory ventilator fed with the desired gas(es).

Typically, the gas mixture administered is essentially made up of nitrous oxide, oxygen and nitrogen, the proportion by volume of nitrous oxide being between 15% and 45% and that of oxygen being typically between 20% and 50%, in particular at least 21% of oxygen.

The administration time ranges, depending on the patient, between a few minutes and several hours, for example between 5 minutes and 4 or 5 hours. The inhalation can be repeated several times in a row, for example several days in a row.

The concentration and/or the administration time most suitable for a given patient can be selected empirically by the care staff, for example according to the patient's state of health or physical condition, the severity of the pain, the sex of the patient, the age of the patient, etc.

The gas mixture used according to the invention is not only effective at low concentrations (<50 vol %) for treating pain hypersensitivity, mainly hyperalgesia and allodynia, but also generates few or no adverse effects in the patients.

Indeed, nitrous oxide is considered, in the medical environment, to be a safe gas provided that its concentration is below approximately 50%, as recalled by the documents D. Annequin et al. Fixed 50% nitrous oxide oxygen mixture for painful procedures: A French survey. Pediatrics 105, E47 (2000); P. Onody, Safety of inhalation of a 50% nitrous oxide/oxygen premix: a prospective survey of 35 828 administrations, Drug Saf 29, 633-640 (2006); and J. T. Knape, Nitrous oxide not unsafe but used less often, Ned. Tijdschr. Geneeskd. 150, 1053-1054 (2006).

Furthermore, nitrous oxide is a gas which has strong N-methyl-D-aspartate (NMDA) receptor antagonist properties, as described by the document S. K. Georgiev et al., Nitrous oxide inhibits glutamatergic transmission in spinal dorsal horn neurons, Pain 134, 24-31 (2008); by the document P. Nagele et al., Nitrous oxide requires the N-methyl-D-aspartate receptor for its action in Caenorhabditis elegans, Proc. Natl. Acad. Sci. U.S.A 101, 8791-8796 (2004), and the document V. Jevtovic-Todorovic et al., Nitrous oxide (laughing gas) is an NMDA antagonist, neuroprotectant and neurotoxin, Nat. Med. 4, 460-463 (1998).

In addition, preclinical data reported in the documents B. Bessiere et al., Nitrous oxide prevents latent pain sensitization and long-term anxiety-like behavior in pain and opioid-experienced rats, Neuropharmacology 53, 733-740 (2007); and P. Richebe et al. Nitrous oxide revisited: evidence for potent antihyperalgesic properties; Anesthesiology 103, 845-854 (2005) have shown a preventive effect of N₂O on post-operative hyperalgesia, in particular that induced by opioids.

However, in the context of the present invention, the gas mixture containing nitrous oxide is no longer used preventively, as in the case of the prevention of post-operative hyperalgesia, but curatively in order to treat patients with chronic pain and thus to obtain a delayed pain hypersensitivity reduction for said patients.

The use, according to the invention, of this nitrous oxide-based mixture will make it possible to reduce or abolish, in a sustained manner, the pain hypersensitivity, mainly hyperalgesia and allodynia, which characterizes patients with chronic pain.

Among the chronic pain targeted by the invention, mention may be made of:
neuropathic pain, i.e. pain related to nerve damage, for instance post-shingles pain, pain caused by diabetic neuropathies, central pain, post-stroke pain, pain caused by multiple sclerosis, neuropathic pain caused by medullary traumas, AIDS-related neuropathic pain, cancer-related neuropathic pain, chemotherapy-related neuropathic pain, post-operative neuropathic pain, etc.,
dysfunctional pain, i.e. with the absence of known damage, such as pain related to fibromyalgia, to an irritable bowel, to cephalalgia, etc.,
inflammatory pain, i.e. inflammatory damage, such as pain related to arthrosis, to arthritis, to a herniated disk, etc.

At the neurobiological level, underlying these three major types of chronic pain is a process of pain sensitization which is reflected by intense hyperalgesia and allodynia which are not very sensitive to the current therapeutic compounds.

The present invention using an inhalable gas mixture containing nitrous oxide therefore aims to reduce or abolish these pain hypersensitivities, i.e. hyperalgesia and allodynia, in order to improve the rehabilitation of “pain” patients.

In other words, the present invention is therefore based on the use of a therapeutic gas mixture containing less than 45% by volume of N₂O for producing an inhalable medicament with curative effects that is intended for treating chronic pain, in particular hyperalgesia and allodynia.

Nitrous oxide (N₂O) in a proportion by volume of less than 50% can therefore be used in the context of a therapeutic treatment method in which the N₂O is administered to a mammal, in particular a human, by inhalation, in order to carry out a curative treatment for chronic pain therein.

### EXAMPLES

In order to demonstrate the curative efficacy of the N₂O-based gas according to the invention, trials were carried out with several gas mixtures containing various concentrations of N₂O, as given in table 1 below.

TABLE 1

| Contents | | | |
|---|---|---|---|
| (% by volume) | N₂O | O₂ | N₂ |
| Trial 1 | 12.5% | 50% | 37.5% |
| (according to the | | | |
| invention) | | | |
| Trial 2 | 25% | 50% | 25% |
| (according to the | | | |
| invention) | | | |
| Trial 3 | 35% | 50% | 15% |
| (according to the | | | |
| invention) | | | |
| Trial 4 (comparative) | 50% | 50% | / |

The trials were carried out in the following way.

Materials and methods

A mononeuropathy is induced in male Sprague-Dawley rats (8 per group) by sciatic nerve constriction. More specifically, on D0, the rats are anesthetized with halothane and the sciatic nerve (injured hind paw side) is prepared using chromic catgut, 4 filament. Neuropathic pain lasting more than 40 days is thus induced.

On D7, the rats are placed in a hermetic chamber where they will inhale, for 75 minutes, the test gas mixture or medical air in the control rats (Sham). Repeated exposures to the mixtures take place on D7, D8, and D9.

Other rats are subjected to comparative treatments with conventional painkilling products, namely ketamine (3×10 mg/kg, sc) and morphine (1 mg/kg, sc).

The nociceptive threshold is then measured via a vocalization test in response to a mechanical stimulus (Randall-Selitto).

The data obtained per measurement are expressed as mean (±standard deviation). The level of significance is set starting from P<0.05.

### Results

FIG. 1 represents the persistent reduction in pain hypersensitivity (˜40%) on the side of the injured hind paw following treatment with nitrous oxide at 50% by volume (trial 3).

In addition to its well-known analgesic effect, nitrous oxide at 50% made it possible to reduce, in the neuropathic rat, the pain hypersensitivity by approximately 40% in the injured hind paw. This decrease in pain hypersensitivity is observed for more than 30 days.

By way of comparison, morphine, which is an opioid analgesic conventionally used in the hospital environment, causes an analgesic effect only for 30 minutes (D14) without a delayed effect (following days), as is the case with nitrous oxide.

Trial 3 shows that the treatment with the gas mixture containing 50% of nitrous oxide is much more beneficial than morphine with regard to reducing neoropathic pain.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 illustrates the persistent reduction in pain hypersensitivity (˜40%) on the side of the injured hind paw following treatment with nitrous oxide at 50% by volume;

FIG. 2 illustrates the abolishing of pain hypersensitivity in the non-injured hind paw following treatment with nitrous oxide at 50%;
FIG. 3 illustrates the analgesic effect with the anti-hyperalgesic effect of nitrous oxide at 50%;

FIG. 4 illustrates the effect of 50% N₂O with ketamine;

FIGS. 5A and 5B illustrate delayed anti-sensitization effect of mixtures A, B and C and also of a mixture containing 50% N₂O and of air (control) on a rat hind paw injured by loose constriction (CCI) of the sciatic nerve;

FIGS. 6A and 6B illustrate delayed anti-sensitization effect of mixtures A, B and C and also of a mixture containing 50% N₂O and of air (control) on a rat hind paw injured by loose constriction (CCI) of the sciatic nerve; and

FIGS. 7A and 7B illustrate use of a mixture containing 50% N2O and for air (control) on a rat hind paw injured by loose constriction (CCI) of the sciatic nerve.

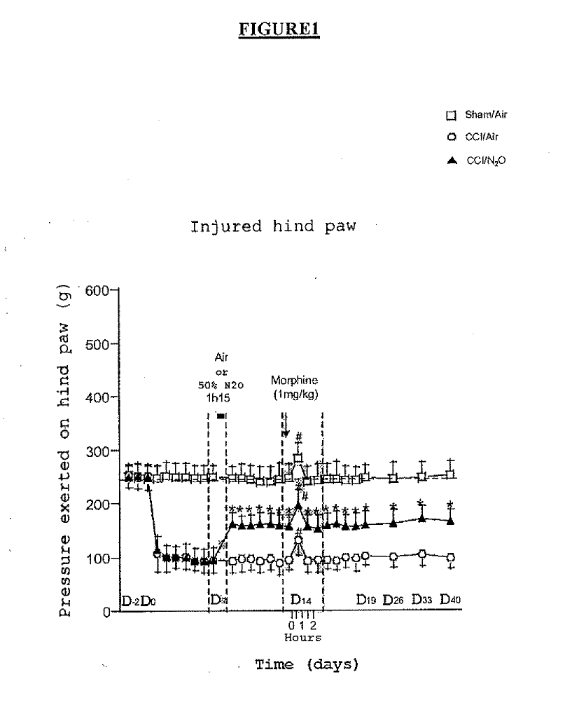

In FIGS. 1 and 2, the following abbreviations are used:

Sham: control. These control neuropathic rats have undergone the same manipulations as the neuropathic rats (anesthesias, surgery, etc.), but with no ligature of the sciatic nerve which induces the neuropathy. The objective is to vary just one parameter at a time (neuropathy or no neuropathy; breathing air or N₂O).

CCI (for chronic constriction injury): sciatic nerve constriction. This is a name that is well established in the literature for denoting the neuropathic animal model used (CCI model). In the present case, CCI/N₂O denotes the group of neuropathic rats treated with N₂O, while CCI/Air denotes the group of neuropathic rats breathing air. These two groups make it possible to deduce the effect of the N₂O in the neuropathic rat.

FIG. 2 shows, for its part, the abolishing of pain hypersensitivity in the non-injured hind paw following treatment with nitrous oxide at 50%. In parallel to its effect in the injured hind paw, the 50% nitrous oxide abolishes the hypersensitivity in the non-injured (contralateral) hind paw, thereby demonstrating a central action of the gas mixture with nitrous oxide at 50% by volume.

In addition, these data suggest that N₂O, by virtue of its action on the central system, could reduce pain hypersensitivity irrespective of the etiology of the chronic pain, namely neuropathic pain, dysfunctional pain, inflammatory pain, provided that a central sensitizing component is present.

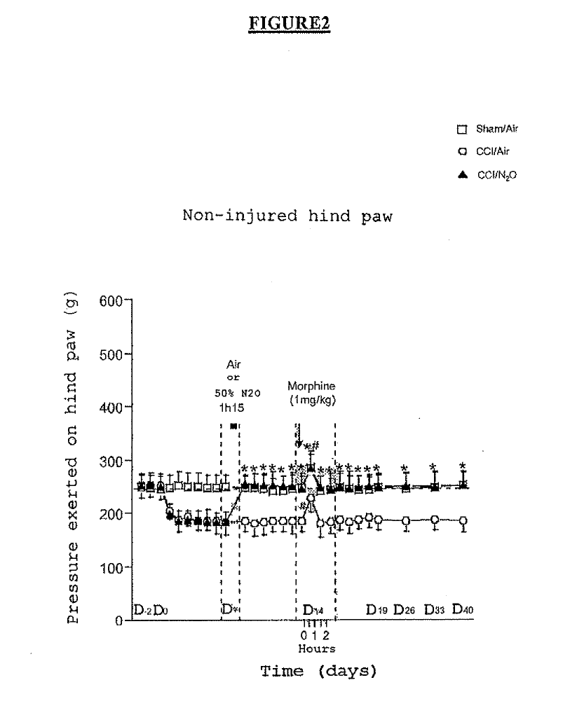

FIG. 3 compares the analgesic effect with the anti-hyperalgesic effect of nitrous oxide at 50%. As is seen, the anti-hyperalgesic (anti-sensitization) effect of N₂O is independent of its analgesic effect. Specifically, blocking the analgesic effect of the 50% nitrous oxide (D7) with naltrexone, which is an opioid receptor antagonist, does not modify its beneficial anti-sensitization effect on the following days (black triangle in FIG. 3).

In other words, the delayed beneficial effect, i.e. the beneficial effect during the days following the treatment with N₂O, and long-term beneficial effect of N₂O at 50% is independent of its analgesic effect.

In FIG. 3, the abbreviation “Nal” is used for naltrexone and the abbreviation “Sal” means saline.

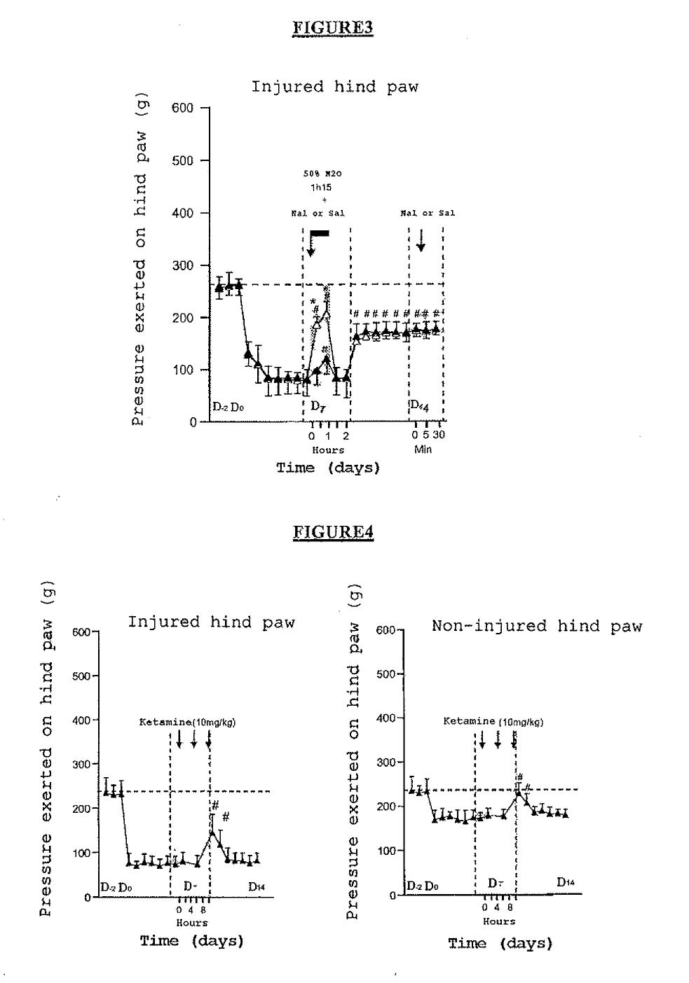

FIG. 4 compares the effect of 50% N₂O with ketamine, which is an NMDA receptor antagonist used in the hospital environment but the use of which remains very limited in humans owing to its considerable adverse effects, in particular psychodysleptic effects.

As is seen, the effect of ketamine is limited to 2 days compared with the sustained effect, of greater than 30 days, obtained with a treatment with 50% N₂O, which does not, moreover, exhibit the adverse effects of ketamine.

Finally, these preclinical data have shown an unexpected effect of a treatment with 50% nitrous oxide since this gas is capable of significantly reducing, in a sustained manner (>1 month), chronic pain of neuropathic type. By comparison, the effect of morphine is limited to a few minutes following its administration, without any anti-hyperalgesic long-term effect. The anti-hyperalgesic effect of an NMDA receptor antagonist such as ketamine is, for its part, limited to 2 days.

Thus, in addition to its analgesic effect, N₂O, via its anti-hyperalgesic properties, could represent a particularly beneficial therapeutic strategy in the treatment of patients with chronic pain, in particular in patients in whom central mechanisms of pain sensitization predominate.

These data obtained with a gas mixture containing 50% of N₂O also suggest that lower concentrations of N₂O, i.e. concentrations below 50% by volume, should have the same anti-sensitization beneficial effects, i.e. of delayed pain hypersensitivity reduction, while at the same time reducing the “acute” sedative/analgesic effect of nitrous oxide.

In fact, at N₂O concentrations of less than 50% by volume, i.e. “sub-anesthetic” concentrations, only a slight amnesia and few sedative and/or analgesic effects are present in human beings, as illustrated in table 2 below, which allows said gas mixture to be easily used without any major risk outside the hospital, in particular at home.

TABLE 2

| N₂O | | | | |
|---|---|---|---|---|
| concentration | Psychic | | | |
| (vol. %) | effects | Amnesia | Analgesia | Contact |
| 5-25% | slight | none | none | present |
| | sedation | | | |
| 26-45% | drunkenness | slight | weak | present |
| 45-65% | drowsiness | complete | moderate | present |

| | |
|---|---|
| 66-85% | Patient unconscious |

In order to demonstrate the efficacy of N₂O concentrations below 50% by volume, supplementary trials were carried out in neuropathic rats, according to the same protocol as above, with gas mixtures containing N₂O, oxygen and, optionally, nitrogen, namely (% by volume):
mixture A: 12.5% N₂O+50% O₂+37.5% N₂
mixture B: 25% N₂O+50% O₂+25% N₂
mixture C: 35% N₂O+50% O₂+15% N₂

To The results obtained show that the delayed anti-sensitization effect is more marked with mixture C than with mixtures A and B. In other words, it is preferable to use N₂O concentrations above 15%, advantageously above 25%, but below or equal to 45%. This is because, since these N₂O concentrations are below 50%, the harmful sedative side effects are much less than with the mixture containing 50% N₂O previously tested, as summarized by table 2 above.

In addition, these trials also showed that it is advantageous to carry out several successive N₂O inhalations for several days in a row, for example one inhalation of 1 hour 15 min for 3 successive days. In fact, carrying out several inhalations for several days in a row makes it possible to increase the desired effect of delayed pain hypersensitivity reduction.

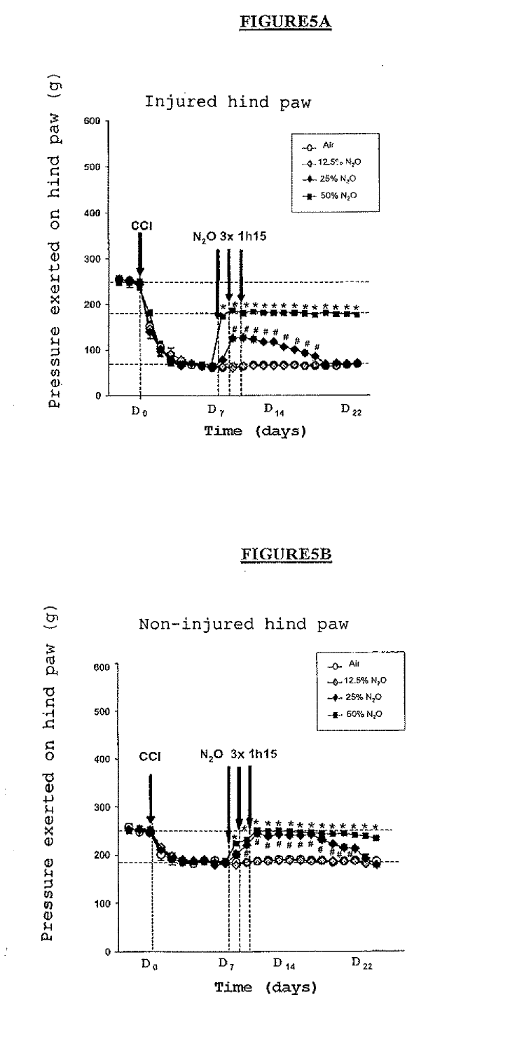

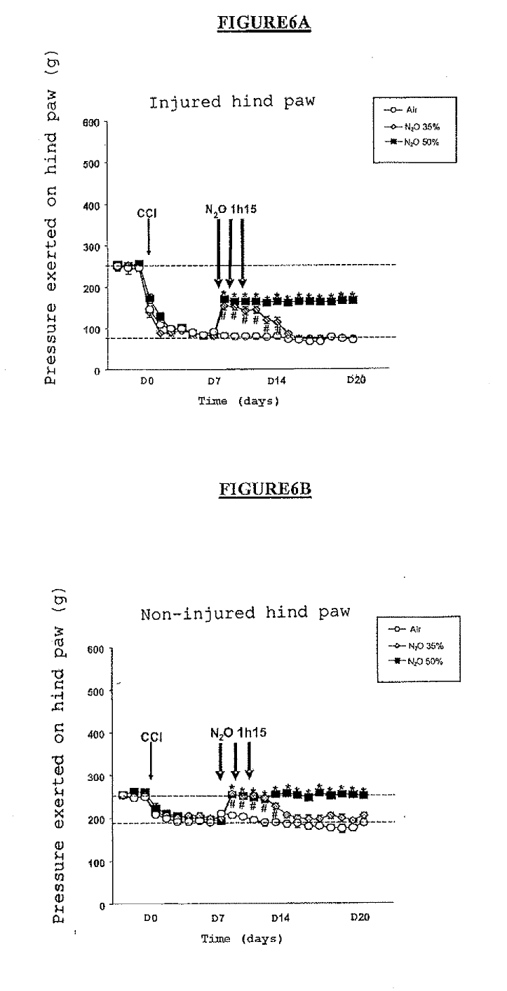

This is illustrated in FIGS. 5A, 5B, 6A and 6B which represent the delayed anti-sensitization effect of mixtures A, B and C and also of a mixture containing 50% N₂O and of air (control) on a rat hind paw injured by loose constriction (CCI) of the sciatic nerve (FIGS. 5A, 6A) and on a non-injured rat hind paw (FIGS. 5B, 6B), to which mechanical pressures are applied in order to measure the pain threshold.

As is seen, administering N₂O, 3 times in a row (1 administration per day) for 1 h 15 min at each inhalation, makes it possible to obtain a long-lasting delayed pain hypersensitivity reduction, that occurs and that can be observed approximately 24 h after the first inhalation and that continues to occur for close to 1 week after the final inhalation for mixtures B and C and the 50% mixture. On the other hand, mixture A (12.5% N₂O) did not, in this case, show any significant difference with the control (air), which confirms that it is preferable to use N₂O concentrations of at least 15% by volume in rats.

Furthermore, these trials also showed that it is advantageous to carry out an N₂O inhalation of greater than 30 min, for example 1 h. In fact, carrying out an inhalation of more than 30 min makes it possible to increase the desired anti-sensitization effect.

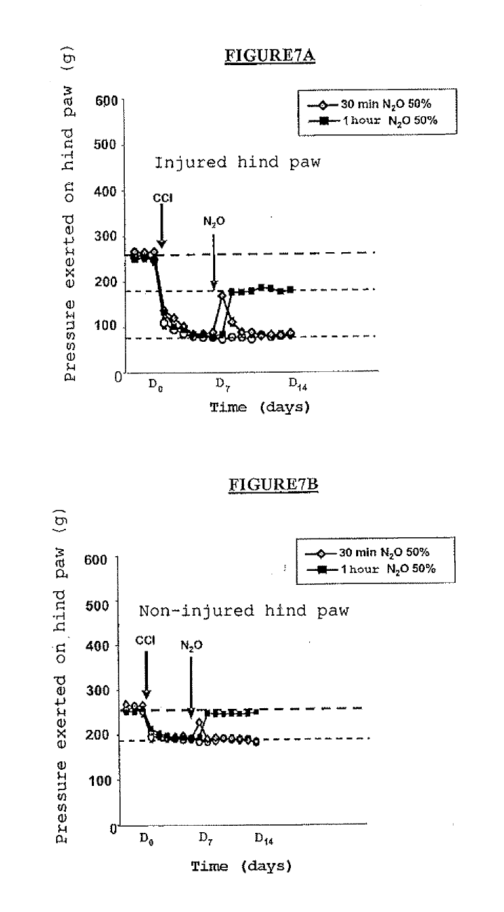

This is illustrated in FIGS. 7A and 7B for a mixture containing 50% N₂O and for air (control) on a rat hind paw injured by loose constriction (CCI) of the sciatic nerve (FIG. 7A) and on a non-injured rat hind paw (FIG. 7B), to which mechanical pressures are applied in order to measure the pain threshold.

As is seen, administering N₂O for 1 h makes it possible to obtain a long-lasting delayed pain hypersensitivity reduction, i.e. anti-sensitization effect, that occurs and that can be observed approximately 24 h after the first inhalation and that continues to occur for close to 1 week after the final inhalation for a 50% mixture.

Hence, according to the invention, use is preferentially made of a gas mixture containing nitrous oxide (N₂O) in a proportion by volume of below 50%, typically below 48%, advantageously between 15% and 45% by volume, as an inhalable medicament for the curative treatment of chronic pain in a mammal, the gas mixture being administered for a period of time sufficient to obtain a delayed pain hypersensitivity reduction, i.e. a delayed anti-sensitization effect, that can be observed at least 6 hours, in general from at least 12 to 24 hours, after the end of the inhalation of the gas mixture by said mammal, in particular in human beings.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1-15. (canceled)

16. A method of inducing a delayed pain hypersensitivity reduction in a subject for the treatment of chronic pain comprising the step of administering by inhalation a gas mixture containing a proportion of nitrous oxide (N₂O) of between 15% and 45% for a period of time sufficient to obtain a delayed pain hypersensitivity reduction that can be observed at least 6 hours after the end of the inhalation of the gas mixture by said subject.

17. The method of claim 16, wherein the subject is a human being.

18. The method of claim 16, wherein the proportion of N₂O in the gas mixture is 20% to 45% by volume.

19. The method of claim 16, wherein the gas mixture is administered for a period of time sufficient to also obtain an analgesic effect in the subject during at least a part of the period of administration by inhalation of the gas mixture.

20. The method of claim 19, wherein the gas mixture is administered for a period of time of at least 20 minutes.

21. The method of claim 16, wherein the delayed anti-sensitization reduction can be observed at least 12 hours after the end of the inhalation of the gas mixture.

22. The method of claim 16, wherein the delayed anti-sensitization reduction can be observed at least 24 hours after the end of the inhalation of the gas mixture.

23. The method of claim 16, wherein the gas mixture also contains between 20% and 60% by volume of oxygen.

24. The method of claim 16, wherein the gas mixture is made up of 15% to 45% by volume of N₂O and at least 20% by volume of oxygen, and further contains one ore more of gaseous xenon, argon, helium, neon, krypton or nitrogen.

25. The gas mixture of claim 16, wherein the gas mixture is made up of 15% to 45% by volume of N₂O and 21% to 60% by volume of oxygen, with the balance, if any, being nitrogen.

26. The gas mixture of claim 16, wherein the chronic pain is of neuropathic origin selected from post-shingles pain, pain caused by diabetic neuropathies, central pain, post-stroke pain, pain caused by multiple sclerosis, neuropathic pain caused by medullary traumas, neuropathic pain related to AIDS or to the treatment thereof, cancer-related neuropathic pain, chemotherapy-related neuropathic pain, neuropathic pain related to a surgical act which has damaged a nerve pathway and post-operative neuropathic pain.

27. The method of claim 16, wherein the chronic pain is of an inflammatory origin due to arthrosis, arthritis, cancer or a herniated disk.

28. The method of claim 16, wherein the gas mixture is administered in combination with an analgesic.

29. The method of claim 16, wherein the pain is of a dysfunctional origin due to fibromyalgia, to irritable bowel syndrome or to cephalalgia.

30. The method of claim 16, wherein the delayed pain hypersensitivity is selected from allodynia and hyperalgesia.
